(19) United States
(12) Patent Application Publication
(10) Pub. No.: US 2016/0152717 A1
(43) Pub. Date: Jun. 2, 2016

(54) METHODS FOR TREATING DRY EYE DISEASE BY ADMINISTERING AN IL-6R ANTAGONIST

(71) Applicant: REGENERON PHARMACEUTICALS, INC., Tarrytown, NY (US)

(72) Inventors: Jingtai CAO, White Plains, NY (US); Ying HU, Scarsdale, NY (US); Carmelo ROMANO, Tarrytown, NY (US); Stanley J. WIEGAND, Hopewell Junction, NY (US)

(73) Assignee: REGENERON PHARMACEUTICALS, INC., Tarrytown, NY (US)

(21) Appl. No.: 14/955,908

(22) Filed: Dec. 1, 2015

Related U.S. Application Data
(60) Provisional application No. 62/086,216, filed on Dec. 2, 2014.
(60) Provisional application No. 62/139,037, filed on Mar. 27, 2015.

(51) Int. Cl. C07K 16/28; A61K 9/00; A61K 45/06; A61K 39/395; A61K 38/13

(52) U.S. Cl. CPC C07K 16/2866; A61K 39/3955; A61K 38/13; A61K 45/06; A61K 9/0048; A61K 9/0014

(57) ABSTRACT

The present invention provides methods for treating dry eye disease by administering an IL-6R antagonist to a subject in need thereof.

### CROSS REFERENCE TO RELATED APPLICATIONS

This application claims the benefit under 35 U.S.C. §119(e) of U.S. provisional application Nos. 62/086,216 filed on Dec. 2, 2014 and 62,139,037 filed on Mar. 27, 2015, the disclosures of which are herein incorporated by reference in their entireties.

### FIELD OF THE INVENTION

The present invention relates to therapies for the treatment of ocular diseases and disorders. More specifically, the present invention relates to the use of IL-6 receptor antagonists to treat dry eye disease.

### BACKGROUND

Dry eye disease refers to a variety of conditions associated with abnormalities in the tear film and insufficient lubrication and/or moisture in the eye. Symptoms of dry eye disease include dryness, scratching, itching, burning, irritation, and a sandy-gritty feeling in the eye. Dry eye disease may also result in visual disturbance and tear film instability, with the potential for damage to the ocular surface. Dry eye disease may be associated with an increase in tear osmolality.

Although various over-the-counter and prescription treatments are available to treat dry eye disease, there remains a need in the art for new treatment options that more directly address the underlying biological causes of the disease. Accordingly, an unmet need exists in the art for novel therapeutic approaches for the treatment of dry eye disease.

### BRIEF SUMMARY OF THE INVENTION

The present invention provides methods for treating or preventing dry eye disease. The methods of the present invention comprise administering an interleukin-6 receptor (IL-6R) antagonist to a subject in need thereof.

The methods of the present invention include administration of an IL-6R antagonist via a variety of routes, including systemic (e.g., intravenous or subcutaneous), intravitreal, or topical administration (e.g., via direct application to the eye in the form of eye drops, gels, ointments, etc.), or via administration routes such as subconjunctival injection, punctual plugs, sub-palpebral depots, and ocular surface depots (e.g., those embedded in contact lenses or other ocular surface adherent devices), as well as other administration routes known in the art and described elsewhere herein.

The methods of the present invention also comprise administering an IL-6R antagonist to a patient in combination with an additional therapeutically active agent. For example, the IL-6R antagonist may be administered in combination with other medications or active components that are known to improve or alleviate one or more symptoms of dry eye disease. The additional therapeutically active agent(s) may be administered in a single formulation with the IL-6R antagonist, or alternatively, the additional therapeutically active agent(s) may be administered to the subject in a formulation or dosage form that is separate and distinct from the formulation or dosage form comprising the IL-6R antagonist.

According to certain embodiments of the methods of the present invention, the IL-6R antagonist may be an anti-IL-6R antibody. Exemplary anti-IL-6R antibodies that may be used in the context of the present invention include, e.g., sarilumab, tocilizumab, or antibodies that compete therewith for binding to IL-6R.

Other embodiments of the present invention will become apparent from a review of the ensuing detailed description.

### BRIEF DESCRIPTION OF THE FIGURES

FIG. 1 shows the tear production results in naïve animals, sham-treated animals, or animals treated with benzalkonium chloride (BAC), scopolamine (Scop), or a combination of BAC and Scop (BAC+Scop), at days −3 (baseline), 7, 14, 21 and 28.

FIG. 2 shows the corneal fluorescein stain score in naïve animals, sham-treated animals, or animals treated with benzalkonium chloride (BAC), scopolamine (Scop), or a combination of BAC and Scop (BAC+Scop), at days −3 (baseline), 7, 14, 21 and 28.

FIG. 3 shows corneal blood vessel skeletonization length in naïve animals, sham-treated animals, or animals treated with benzalkonium chloride (BAC), scopolamine (Scop), or a combination of BAC and Scop (BAC+Scop), at week 4.

FIG. 4 shows corneal lymphatic vessel skeletonization length in naïve animals, sham-treated animals, or animals treated with benzalkonium chloride (BAC), scopolamine (Scop), or a combination of BAC and Scop (BAC+Scop), at week 4.

FIG. 5 shows the CD45+ percentage of live cells, as determined by FACS from the extraorbital lacrimal glands from naïve animals, sham-treated animals, or animals treated with benzalkonium chloride (BAC), scopolamine (Scop), or a combination of BAC and Scop (BAC+Scop), at week 4.

FIG. 6 shows the tear production results in untreated animals, control treated animals (mFc), and animals treated with an anti-mIL-6R antibody at 10 mg/kg, 35 mg/kg and 100 mg/kg, at days −3, 7, 14, 21, and 28, in the dry eye model described in Example 1 herein.

FIG. 7 shows the corneal fluorescein stain score in untreated animals, control treated animals (mFc), and animals treated with an anti-mIL-6R antibody at 10 mg/kg, 35 mg/kg and 100 mg/kg, at days −3, 7, 14, 21, and 28, in the dry eye model described in Example 1 herein.

FIG. 8 shows corneal blood vessel skeletonization length in untreated animals, control treated animals (mFc), and animals treated with an anti-mIL-6R antibody at 10 mg/kg, 35 mg/kg and 100 mg/kg, in the dry eye model described in Example 1 herein.

FIG. 9 shows corneal lymphatic vessel skeletonization length in untreated animals, control treated animals (mFc), and animals treated with an anti-mlL-6R antibody at 10 mg/kg, 35 mg/kg and 100 mg/kg, in the dry eye model described in Example 1 herein.

FIG. 10 shows the graphical relationship between lymphatic vessel length (y-axis) and corneal fluorescein staining score (x-axis) in animals treated in accordance with the experiments described in Example 1 herein.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present invention, the preferred methods and materials are now described. All publications mentioned herein are incorporated herein by reference to describe in their entirety.

### Dry Eye Disease

The present invention includes methods for treating or preventing dry eye disease. As used herein, “dry eye disease” (also known as “dry eye syndrome,” “dry eye disorder,” etc.) refers to any disease, condition or affliction characterized by one or more of: (a) decrease in tear production; (b) increase in tear film evaporation; (c) loss of mucous-containing conjunctival goblet cells; (d) desquamation of the corneal epithelium; and/or (e) destabilization of the cornea-tear interface. Dry eye disease may be characterized, according to known clinical criteria, as mild, moderate, moderate-to-severe, and severe dry eye disease. Accordingly, the present invention provides methods of treating any degree of dry eye disease, including mild dry eye disease, moderate dry eye disease, moderate-to-severe dry eye disease, or severe dry eye disease. Dry eye disease may be acute or chronic. Accordingly, the present invention provides methods of treating either acute or chronic dry eye disease. Dry eye disease may also be categorized as either “tear deficient dry eye disease” or “evaporative dry eye disease.” Accordingly, the present invention provides methods of treating tear deficient dry eye disease and/or evaporative dry eye disease.

According to the present invention, “dry eye disease” includes, e.g., age-related dry eye, blepharitis, conjunctivitis, corneal desquamation, corneal infiltrates, epithelial edema, giant papillary conjunctivitis, hypoxia, keratoconjunctivitis sicca (KCS), microbial keratitis, microcysts, ocular cicatrical pemphigoid, Stevens-Johnson syndrome, Sjogren's syndrome, and ulcerative keratitis. “Dry eye disease” also includes dry eye conditions associated with corneal injury, corneal surgery (including LASIK), contact lens usage, infection, nutritional disorders or deficiencies, pharmacologic agents, eye stress, glandular and tissue destruction, exposure to pollutants and environmental conditions (e.g., smog, smoke, excessively dry air), airborne particulates, autoimmune and other immunodeficient disorders, and other conditions that impair or inhibit the ability of an individual to blink. The methods of the present invention may be used to treat or prevent any of the foregoing conditions that fall under the definition of “dry eye disease”.

### Interleukin-6 Receptor Antagonists

The term “interleukin-6 receptor” (IL-6R) means a human cytokine receptor that specifically binds human interleukin-6 (IL-6). Human IL-6 is a cytokine having the amino acid sequence as set forth in NCBI accession number NP000591. Human IL-6R is a protein complex consisting of an IL-6R subunit (also known as the “IL-6R-alpha subunit”) and a GP130 signal transduction subunit. The IL-6R subunit has the amino acid sequence as set forth in NCBI accession number NP000556. As used herein, the expression “interleukin-6 receptor antagonist,” or “IL-6R antagonist,” means any agent which binds to or interacts with IL-6R and inhibits the normal biological signaling function of IL-6 and/or IL-6R in vitro or in vivo.

An exemplary class of molecules that may function as IL-6R antagonists for use in the methods of the present invention include anti-IL-6R antibodies. For example, IL-6R antagonists that can be used in the context of the present invention include, e.g., chimeric, humanized or fully human anti-IL-6R antibodies, or antigen-binding fragments of any of these kinds of antibodies.

The term “antibody”, as used herein, means any antigen-binding molecule or molecular complex comprising at least one complementarity determining region (CDR) that specifically binds to or interacts with a particular antigen (e.g., IL-6R). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As used herein, the expression “anti-IL-6R antibody” also includes multispecific antigen-binding molecules (e.g., bispecific antibodies) wherein at least one binding domain (e.g., “binding arm”) of the multispecific antigen-binding molecule specifically binds IL-6R.

Exemplary anti-IL-6R antibodies that can be used in the context of the present invention include, e.g., the humanized anti-IL-6R antibody tocilizumab (Chugai) (e.g., as set forth in U.S. Pat. No. 5,795,965), and the fully-human anti-IL-6R antibody sarilumab (Regeneron/Sanofi) (e.g., an anti-IL-6R antibody comprising the heavy and light chain variable regions having amino acid sequences SEQ ID NO:19 and SEQ ID NO:27, respectively, as set forth in U.S. Pat. No. 7,582,298). Other IL-6R antagonists that can be used in the context of the methods of the present invention include the anti-IL-6R nanobody referred to as ALX-0061 (Abbvie, Ablynx) (e.g., as set forth in US20100215664), APX007 (Apexigen) (e.g., as set forth in U.S. Pat. No. 8,753,634), second-generation tocilizumab (Chugai) (e.g., as set forth in US 2013/0317203), CytomX anti-IL-6R antibody (e.g., as set forth in WO 2014/052462), Medimmune anti-IL-6:IL6Ra complex antibody (e.g., as set forth in U.S. Pat. No. 8,153,128), and NI-1201 (Novimmune) (e.g., as set forth in U.S. Pat. No. 8,034,344). The disclosures of all of the aforementioned patents and patent application publications are incorporated by reference herein in their entireties.

The present invention also includes the use of anti-IL-6R antibodies that bind to the same epitope as, or compete for binding with any one of the specific anti-IL-6R antibodies mentioned herein.

One can easily determine whether an antibody binds to the same epitope as, or competes for binding with, a reference anti-IL-6R antibody by using routine methods known in the art. For example, to determine if a test antibody binds to the same epitope as a reference anti-IL-6R antibody of the invention, the reference antibody is allowed to bind to an IL-6R protein (e.g., a soluble portion of the IL-6R extracellular domain or cell surface-expressed IL-6R). Next, the ability of a test antibody to bind to the IL-6R molecule is assessed. If the test antibody is able to bind to IL-6R following saturation binding with the reference anti-IL-6R antibody, it can be concluded that the test antibody binds to a different epitope than the reference anti-IL-6R antibody. On the other hand, if the test antibody is not able to bind to the IL-6R molecule following saturation binding with the reference anti-IL-6R antibody, then the test antibody may bind to the same epitope as the epitope bound by the reference anti-IL-6R antibody of the invention. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present invention, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990:50:1495-1502). Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if an antibody competes for binding with a reference anti-IL-6R antibody, the above-described binding methodology is performed in two orientations: In a first orientation, the reference antibody is allowed to bind to an IL-6R protein (e.g., a soluble portion of the IL-6R extracellular domain or cell surface-expressed IL-6R) under saturating conditions followed by assessment of binding of the test antibody to the IL-6R molecule. In a second orientation, the test antibody is allowed to bind to an IL-6R molecule under saturating conditions followed by assessment of binding of the reference antibody to the IL-6R molecule. If, in both orientations, only the first (saturating) antibody is capable of binding to the IL-6R molecule, then it is concluded that the test antibody and the reference antibody compete for binding to IL-6R. As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

### Pharmaceutical Compositions Comprising an IL-6R Antagonist

The present invention includes pharmaceutical compositions comprising an IL-6R antagonist (e.g., anti-IL-6R antibody), and methods of use thereof. The pharmaceutical compositions according to this aspect of the invention may further comprise a pharmaceutically acceptable carrier or diluent. Methods for co-formulating biological therapeutic agents are known in the art and may be used by a person of ordinary skill in the art to make the pharmaceutical compositions of the present invention.

As used herein, the expression “pharmaceutically acceptable carrier or diluent” includes suitable carriers, excipients, and other agents that provide suitable transfer, delivery, tolerance, and the like. Exemplary formulations useful in the context of the present invention can be found in Remington's Pharmaceutical Sciences, Mack Publishing Company, Easton, Pa. Acceptable formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Exemplary pharmaceutical compositions comprising an anti-IL-6R antibody that can be used in the context of the present invention are set forth in, e.g., US 2011/0171241, the disclosure of which is incorporated by reference herein in its entirety.

### Additional Therapeutic Agents

The present invention includes compositions and therapeutic formulations comprising an IL-6R antagonist (e.g., anti-IL-6R antibody) in combination with one or more additional therapeutically active components. The present invention also includes methods of treatment comprising administering such combinations to subjects in need thereof (e.g., in a single dosage form). Similarly, the present invention includes methods of treating dry eye disease in a subject comprising administering an IL-6R antagonist (e.g., anti-IL-6R antibody) in combination with one or more additional therapeutically active components, wherein the one or more additional therapeutically active components is administered to the subject in a separate dosage form.

Additional therapeutically active components that may be combined with and/or administered in combination with an IL-6R antagonist in the context of the present invention include, e.g., one or more of the following: a VEGF antagonist, e.g., a “VEGF-trap” such as aflibercept or other VEGF-inhibiting fusion proteins as set forth in U.S. Pat. No. 7,087,411, an anti-VEGF antibody or antigen binding fragment thereof (e.g., bevacizumab, ranibizumab), a small molecule kinase inhibitor of VEGF receptor (e.g., sunitinib, sorafenib or pazopanib), an anti-VEGF receptor antibody, an anti-PDGFR-beta antibody (e.g., an anti-PDGFR-beta antibody as set forth in US 2014/0193402 [e.g., the antibody referred to as H4H3374N or H4H3094P]), a PDGF ligand antagonist (e.g., an anti-PDGF-BB antibody, an anti-PDGF-DD antibody, an anti-PDGF-CC antibody, an anti-PDGF-AB antibody, or other PDGF ligand antagonist such as an aptamer [e.g., an anti-PDGF-B aptamer such as Fovista™, Ophthotech Corp., Princeton, N.J.], an antisense molecule, a ribozyme, an siRNA, a peptibody, a nanobody or an antibody fragment directed against a PDGF ligand).

Other types of additional therapeutically active components that may be combined with and/or administered in combination with an IL-6R antagonist in the context of the present invention include, e.g., anti-infective agents, antibiotics, antiviral agents, anti-inflammatory drugs, antiallergic agents, vasoconstrictors, vasodilators, local anesthetics, analgesics, intraocular pressure-lowering agents, immunoregulators, anti-oxidants, vitamins and minerals, corticosteroids, steroids, COX inhibitors, cardioprotectants, metal chelators, IFN-gamma, and/or NSAIDs. Specific examples of such additional therapeutically active components include, e.g., silver, iodine, aminoglucosides, quinolones, macrolides, cephems, and sulfa drugs such as sulfamethoxazole, sulfisoxazole, sulfisomidine, sulfadiazine, sulfadimethoxine, sulfamethoxypyridazine, famciclovir, penciclovir, acyclovir, indomethacin, diclofenac, pranoprofen, tiaprofenic acid, tolfenamic acid, prednisolone, dipottasium glycyrrhizinate, allantoin, epsilon-aminocaproic acid, berberine chloride, berberine sulfate, sodium azulenesulfonate, zinc sulfate, zinc lactate, lysozyme chloride, ketotifen, oxatomide, cetirizine, sodium cromoglicate, mequitazine, chlorpheniramine maleate, diphenhydramine hydrochloride, naphazoline, tetrahydrozoline, oxymethazoline, phenylephrine, ephedrines, epinephrine, lidocaine hydrochloride, procaine hydrochloride, dibucaine hydrochloride, cylcosporin A, tacrolimus, vitamin A, vitamin C, vitamin E, vitamin B1, B2, B6, B12, nicotinates, pantothenates, and biotin.

The additional therapeutically active component(s), e.g., any of the agents listed above or derivatives or combinations thereof, may be administered just prior to, concurrent with, or shortly after the administration of an IL-6R antagonist, within the context of the present invention; (for purposes of the present disclosure, such administration regimens are considered the administration of an IL-6R antagonist “in combination with” an additional therapeutically active component). The present invention includes pharmaceutical compositions in which an IL-6R antagonist is co-formulated with one or more of the additional therapeutically active component(s) as described elsewhere herein.

### Drug Delivery and Methods of Administration

The IL-6R antagonist (or pharmaceutical formulation comprising the IL-6R antagonist) may be administered to the patient by any known delivery system and/or administration method. In certain embodiments, the IL-6R antagonist is administered to the patient by ocular, intraocular, intravitreal or subconjunctival injection. In other embodiments, the IL-6R antagonist can be administered to the patient by topical administration, e.g., via eye drops or other liquid, gel, ointment or fluid which contains the IL-6R antagonist and can be applied directly to the eye. Other administration methods that can be used in the context of the present invention include, e.g., administration of an IL-6R antagonist via depots placed on or around the eye, including active agent embedded in a contact lens or other ocular surface adherent device, or in a punctual plug. Other possible routes of administration include, e.g., intradermal, intramuscular, intraperitoneal, intravenous, subcutaneous, intranasal, epidural, and oral.

Various delivery systems are known and can be used to administer the pharmaceutical compositions of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). The composition(s) may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present invention (comprising, e.g., a single therapeutically active agent, or a combination of two or more therapeutically active agents) can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used. In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Dosage

The amount of active ingredient(s) (e.g., IL-6R antagonist) contained within the pharmaceutical compositions of the present invention, and/or administered to a subject according to the methods of the present invention, is generally a therapeutically effective amount. As used herein, the expression “therapeutically effective amount,” in the context of an IL-6R antagonist, means an amount of the therapeutic agent, alone or in combination with another therapeutic agent, that is capable of producing a measureable biological effect in a human or animal subject. Examples of such measurable biological effects include, e.g., detection of the therapeutic molecule in the serum of the subject, detection of relevant metabolic products in a biological sample taken from the subject, a change in the concentration of a relevant biomarker in a sample taken from the subject, an improvement in a sign or symptom of dry eye disease, and/or an improvement in any other relevant therapeutic or clinical parameter.

In the case of an anti-IL-6R antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-IL-6R antibody.

The amount of anti-IL-6R antibody administered to a subject may be expressed in terms of milligrams of antibody per kilogram of subject body weight (i.e., mg/kg). For example, the anti-IL-6R antibody may be administered to a patient at a dose of about 0.0001 to about 10 mg/kg of subject body weight.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Efficacy of an Anti-IL-6R Antibody in a Murine Dry Eye Model

In this Example, a mouse dry eye model was established and the effects of IL-6 receptor antagonism in this model were assessed. The IL-6R antagonist used in these experiments was a mouse monoclonal antibody specific for mouse anti-IL-6 receptor (“anti-mIL6R Ab”). The dry eye model system developed for these experiments involved a combination of aqueous tear deficiency and local cornea inflammation.

### Methods:

Adult, male C57BL/6 mice (Taconic labs) were randomized to naive, sham, benzalkonium chloride (BAC), scopolamine or BAC plus scopolamine groups. For the scopolamine group, mice were subcutaneously implanted with an osmotic pump filled with scopolamine (delivering 2 mg/20 g B.W./day) lasting for 4 weeks. For BAC treatment, 1 μl 0.2% BAC was administered topically on the right eye ocular surface, B.I.D, 2 days/week.

For antibody treatment, mice received BAC plus scopolamine and either anti-mIL6R Ab (10, 35 or 100 mg/kg) or mouse IgG2a (“mFc”) control (33.3 mg/kg) subcutaneously administered twice/week for 4 weeks.

Tear production and corneal fluorescein staining were measured every week for four weeks.

On day 28 after induction of dry eye disease, mice were euthanized and eyeballs were collected in 4% PFA for corneal angiogenesis and lymphangiogenesis. Additionally, extraorbital lacrimal glands were harvested for flow cytometry to measure immune cell infiltration. Cornea was dissected, washed in PBS, blocked for 1 hour at room temperature, stained with LYVE1 and rat-anti-mouse CD31 antibody (Santa Cruz Biotech, SC-71871; 1:100) 4° C. overnight. After washing in PBS, tissue was incubated with secondary antibody conjugates for 2 hours at room temperature and flat-mounted onto glass slides. Images of stained blood and lymphatic vessels were captured using a digital RT SE Spot camera attached to a fluorescence microscope (Nikon Eclipse 80i). Image J software was used for the image analysis. Extraorbital lacrimal glands were kept in 3% FBS till processed for flow cytometry and analysis.

For lacrimal gland dissociation and flow cytometry analysis, extraorbital lacrimal glands were dissected from surrounding tissue, cut to small pieces (1-2 mm in diameter) and incubated with Liberase DL (0.5 mg/ml) for 20 mins at 37° C., mixed by inverting the tubes every 5 mins. EDTA (10 mM) was added to stop the reaction. Tissue was re-suspended with a 1 ml pipet and filtered through a 70um cell strainer. Cell suspension was collected, rinsed with 5 ml FACS buffer, stained for live/dead stain and CD45, analyzed using a BD LSRII machine.

### Results:

### Evaluation of the Dry Eye Model

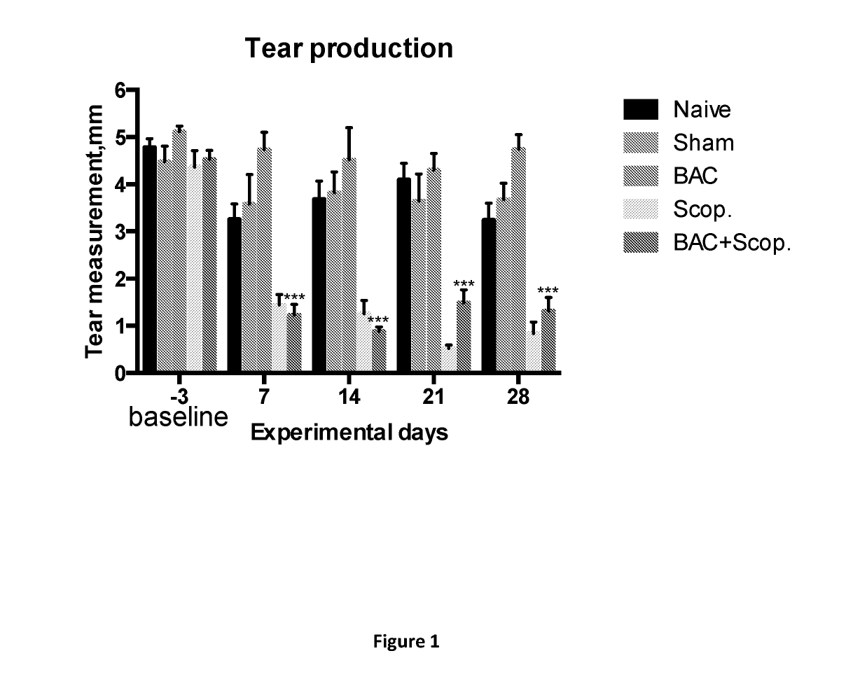

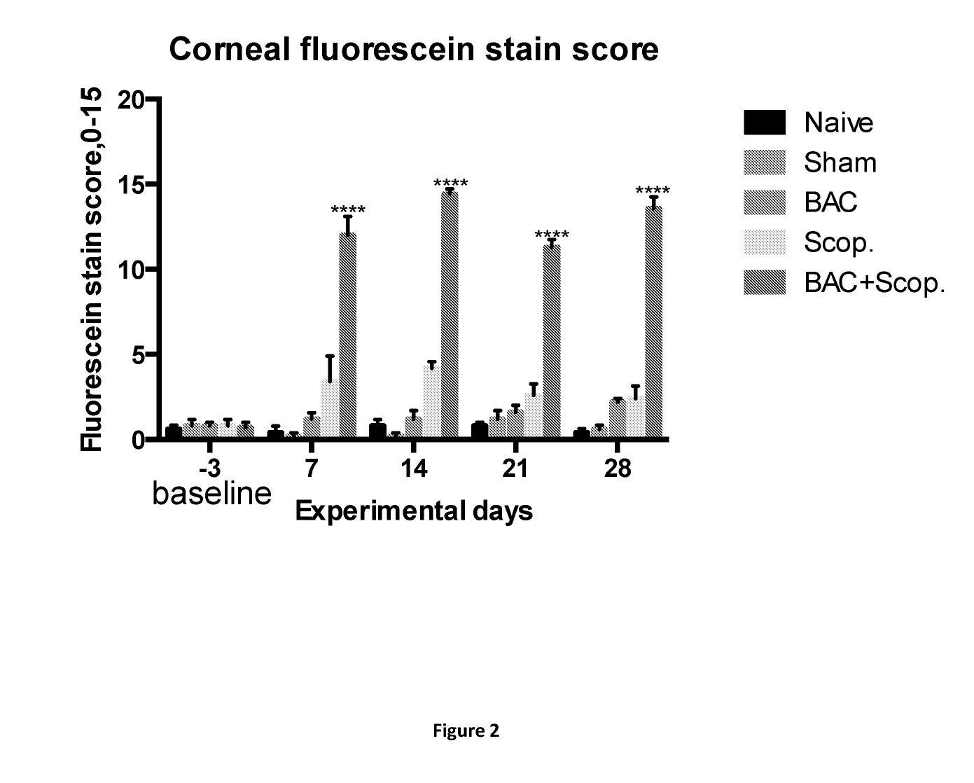

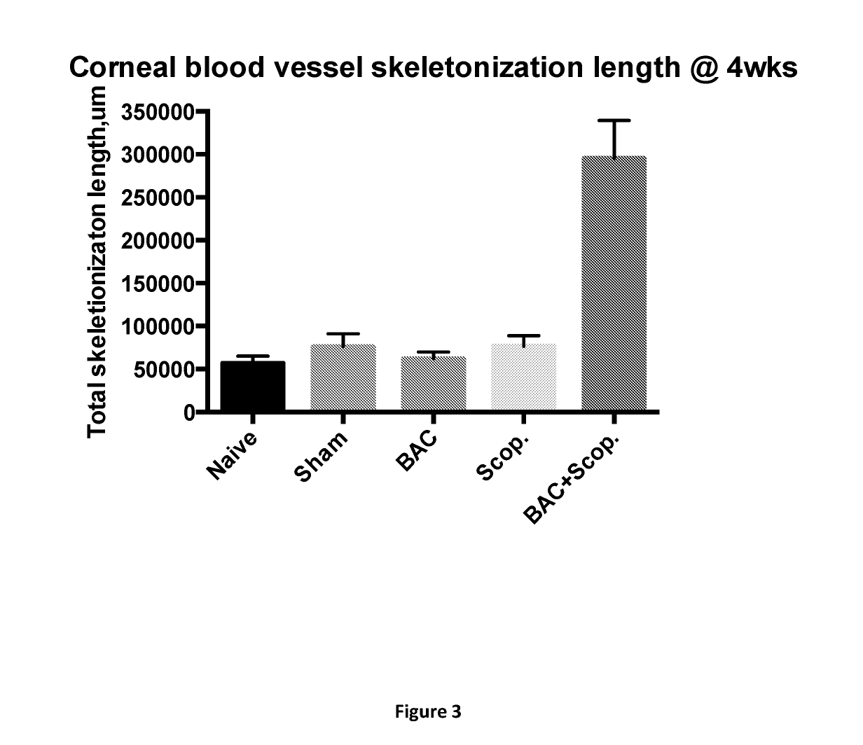

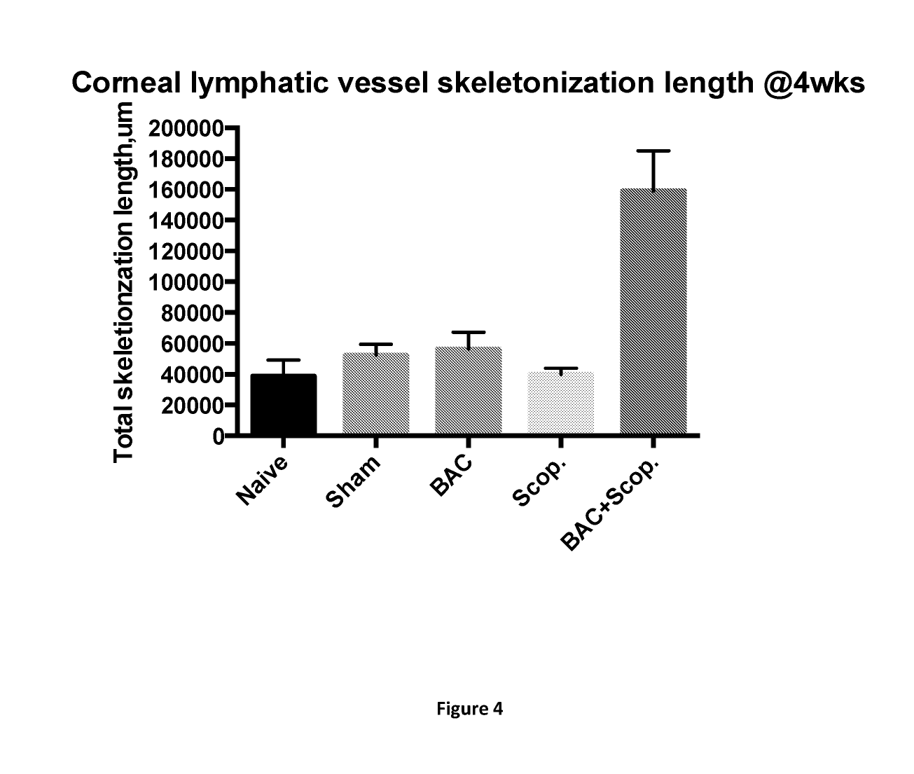

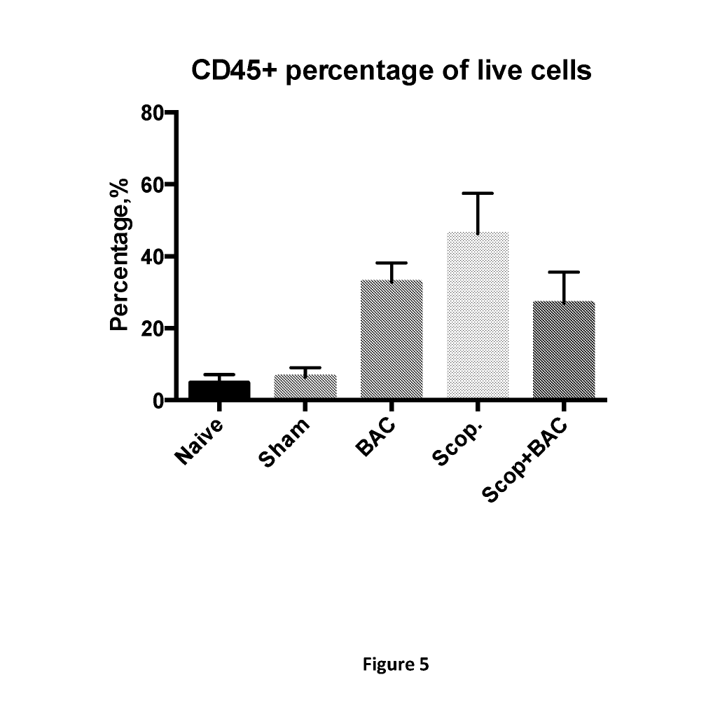

Wild type mice treated with BAC plus scopolamine provided a robust dry eye syndrome. Tear production was inhibited by more than 50% in both scopolamine- and BAC+scopolamine-treated groups. (FIG. 1). Corneal fluorescein staining score was significantly increased in the scopolamine plus BAC group compared to the other groups (p<0.001). (FIG. 2). Corneal angiogenesis and lymphangiogenesis were also significantly increased in this dry eye model. (FIGS. 3 and 4). Interestingly, lymphogenesis but not angiogenesis was significantly correlated with corneal fluorescein staining (p<0.01). CD45, B220, CD4 and CD8 positive cell infiltration were found in the extraorbital lacrimal gland. (See, e.g., FIG. 5 [CD45+ cells]).

### Efficacy of an Anti-IL-6R Antibody in the Dry Eye Model

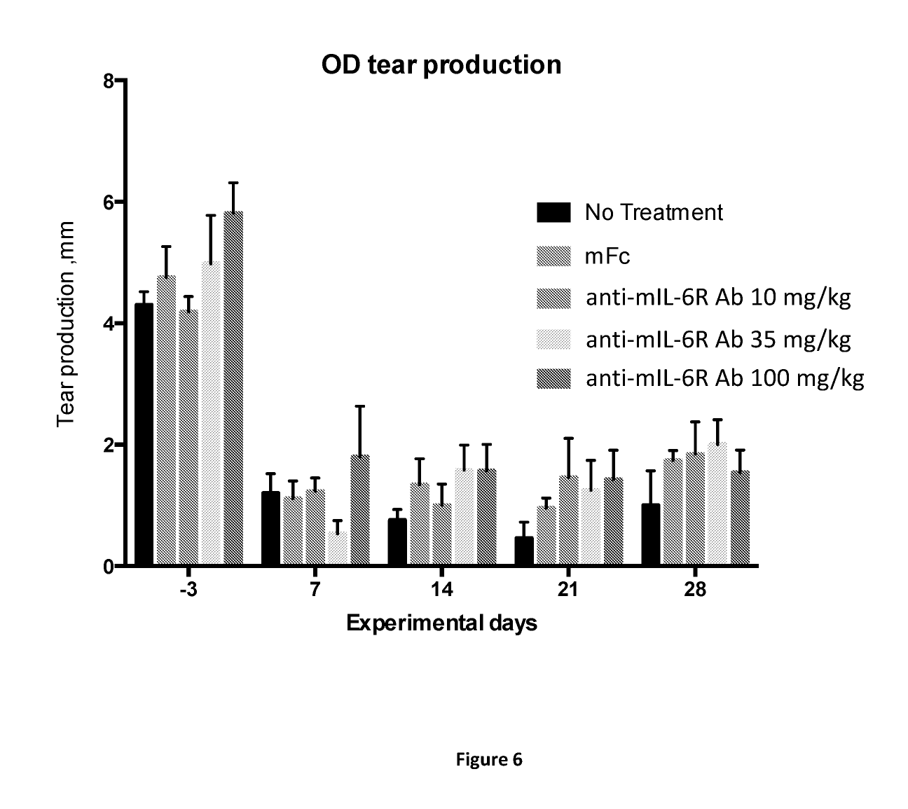

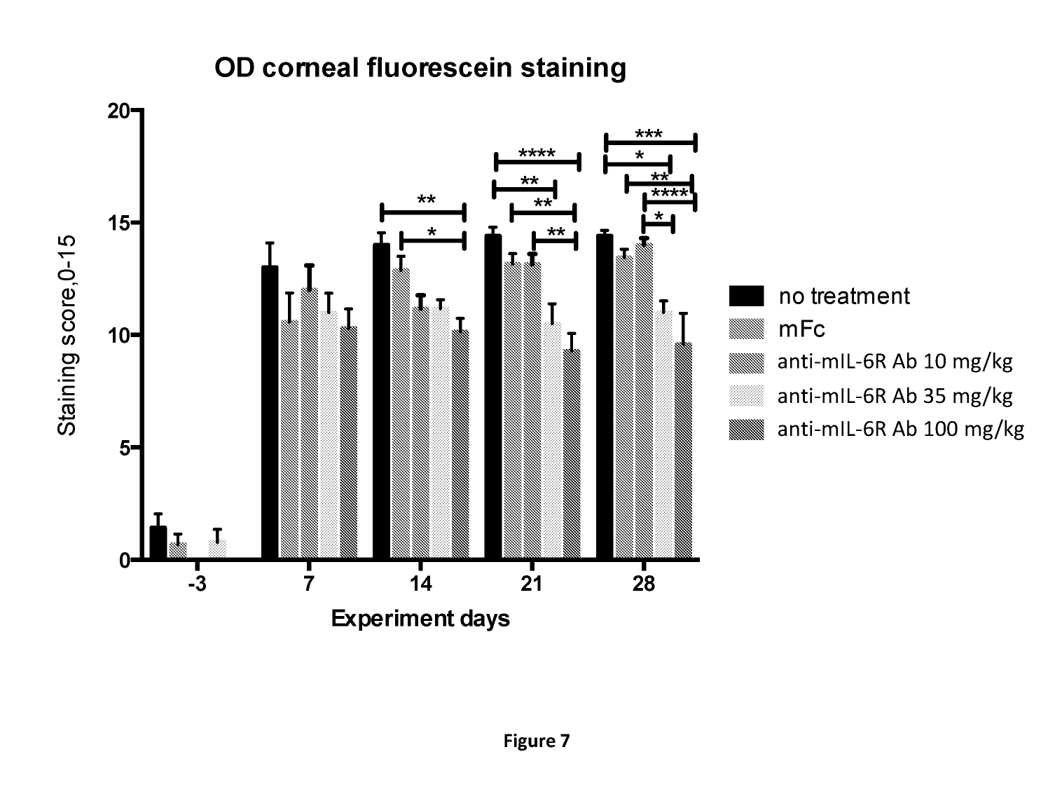

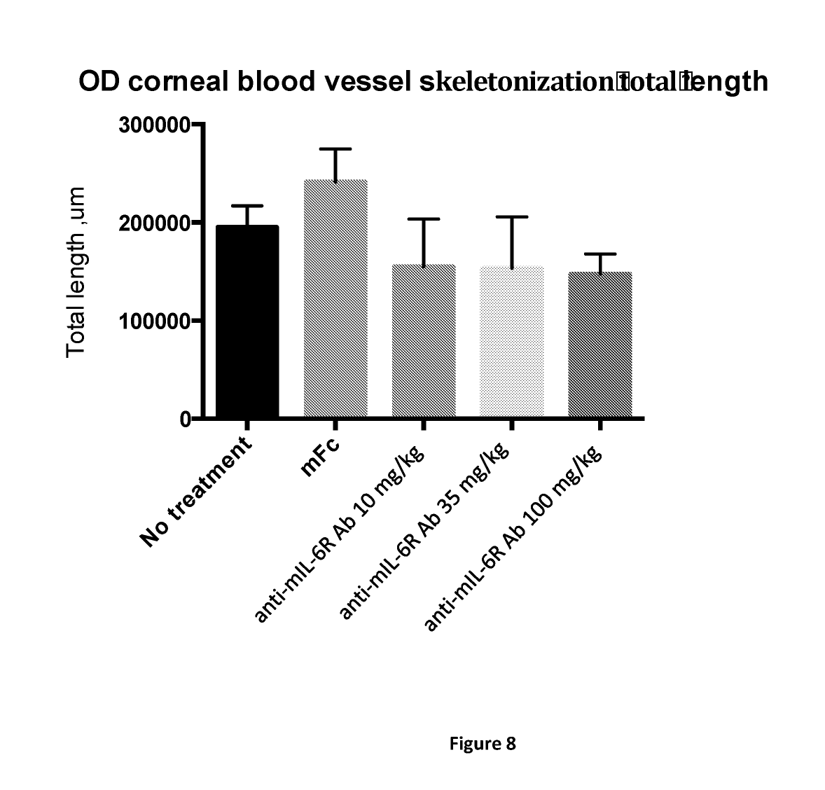

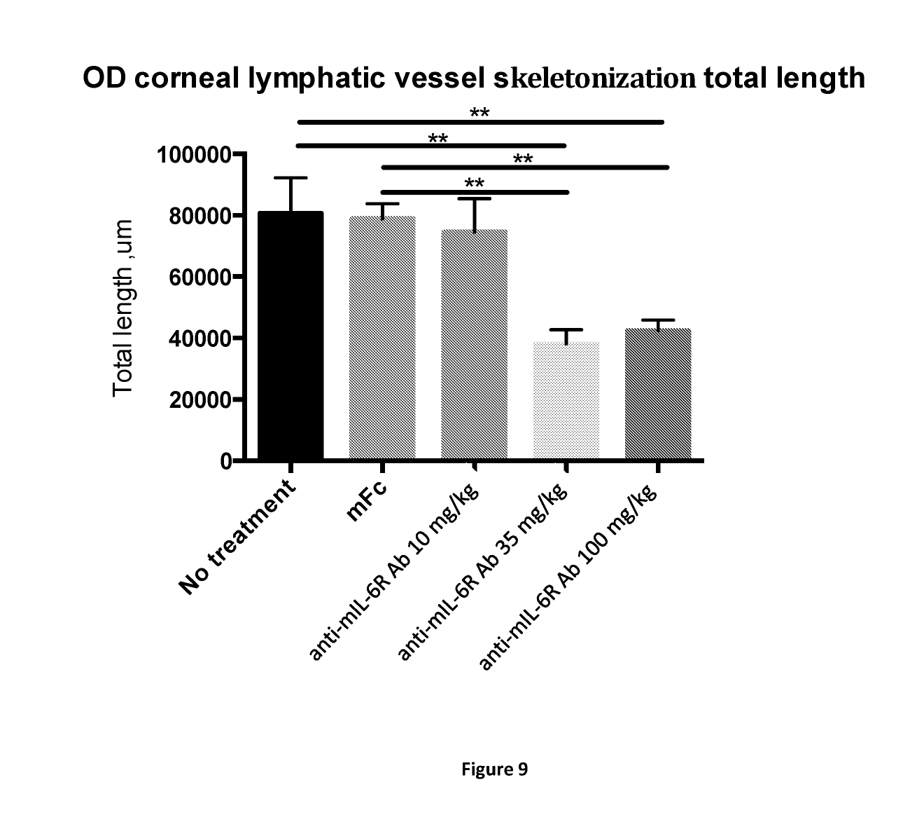

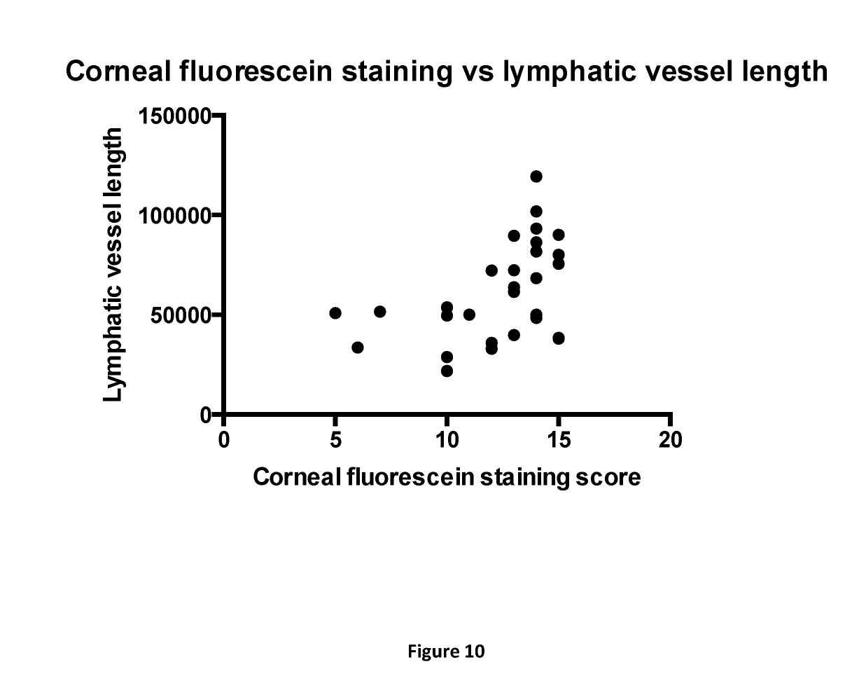

The effects of an anti-IL-6R antibody in the above-described dry eye model, expressed in terms of tear production, corneal fluorescein staining, corneal blood vessel skeletonization, as compared to controls, are shown in FIGS. 6-9. There was no significant difference in body weight or tear production in mFc or anti-IL-6R Ab treated groups on day 28 compared to no treatment control. Compared with mFc, anti-IL-6R treatment dose-dependently and significantly decreased corneal fluorescein staining (30% reduction with 100 mg/kg) at 3 or 4 weeks (FIG. 7), and reduced corneal lymphangiogenesis (50% reduction with 100 mg/kg) at four weeks. (FIGS. 8 and 9). Moreover, corneal fluorescein staining and lymphatic vessel length were found to be highly correlated with one another (p=0.009, Spearman test). (FIG. 10).

Thus, in the dry eye model used in this Example, anti-IL-6R treatment decreased cornea damage in a dose-dependent manner, without affecting body weight or tear production. In addition, anti-IL-6R treatment significantly decreased cornea lymphangiogenesis, but had no effect on cornea blood vessel length.

### CONCLUSION

This Example describes the development of a novel dry eye model with reduced tear secretion and enhanced corneal damage and lymphangiogenesis in cornea and inflammation in lacrimal gland. Systemic administration of anti-IL-6R antibody was shown to alleviate the corneal damage and reduced lymphocyte infiltration in the lacrimal gland. This Example supports the use of anti-IL-6R treatment as a therapeutic strategy in dry eye disease.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method for treating or preventing dry eye disease, the method comprising administering an interleukin-6 receptor (IL-6R) antagonist to a subject in need thereof.

2. The method of claim 1, wherein the subject at the time of or just prior to administration of the IL-6R antagonist has moderate-to-severe dry eye disease.

3. The method of claim 1, wherein the IL-6R antagonist is administered in combination with an additional therapeutically active agent.

4. The method of claim 3, wherein the additional therapeutically active component comprises cyclosporin A.

5. The method of claim 4, wherein the additional therapeutically active component comprises an TNF-alpha antagonist.

6. The method of claim 1, wherein the IL-6R antagonist is an antibody that specifically binds IL-6R or antigen-binding fragment of an antibody that specifically binds IL-6R.

7. The method of claim 6, wherein the IL-6R antagonist is an anti-IL-6R antibody selected from the group consisting of: (a) tocilizumab, (b) sarilumab, (c) an antibody that competes for binding to IL-6R with tocilizumab, (d) an antibody that binds to the same epitope on IL-6R as tocilizumab, (e) an antibody that competes for binding to IL-6R with sarilumab, and (d) an antibody that binds to the same epitope on IL-6R as sarilumab.

8. The method of claim 6, wherein the IL-6R antagonist is administered to the subject systemically.

9. The method of claim 6, wherein the IL-6R antagonist is administered to the subject by ocular, intraocular, intravitreal or subconjunctival injection.

10. The method of claim 6, wherein the IL-6R antagonist is administered to the subject by topical administration.

11. The method of claim 10, wherein the topical administration is via direct application to the subject's eye in the form or eye drops, gel or ointment comprising the IL-6R antagonist, or via ocular surface depots, punctual plugs or sub-palpebral depots.

12. A method for treating or preventing dry eye disease, comprising:
administering an interleukin-6 receptor (IL-6R) antagonist to a subject in need thereof; and
administering cyclosporine to the subject.

13. The method of claim 12, wherein the subject at the time of or just prior to administration of the IL-6R antagonist has moderate-to-severe dry eye disease.

14. The method of claim 12, further comprising is administered an additional therapeutically active agent.

15. A method for treating or preventing dry eye disease, comprising:
administering an interleukin-6 receptor (IL-6R) antagonist to a subject in need thereof; and
administering TNF-alpha antagonist to the subject.

16. The method of claim 12, wherein the IL-6R antagonist is an antibody that specifically binds IL-6R or antigen-binding fragment of an antibody that specifically binds IL-6R.

17. The method of claim 16, wherein the IL-6R antagonist is an anti-IL-6R antibody selected from the group consisting of: (a) tocilizumab, (b) sarilumab, (c) an antibody that competes for binding to IL-6R with tocilizumab, (d) an antibody that binds to the same epitope on IL-6R as tocilizumab, (e) an antibody that competes for binding to IL-6R with sarilumab, and (d) an antibody that binds to the same epitope on IL-6R as sarilumab.

18. The method of claim 16, wherein the IL-6R antagonist is administered to the subject systemically.

19. The method of claim 16, wherein the IL-6R antagonist is administered to the subject by ocular, intraocular, intravitreal or subconjunctival injection.

20. The method of claim 16, wherein the IL-6R antagonist is administered to the subject by topical administration wherein the topical administration is via direct application to the subject's eye in the form or eye drops, gel or ointment comprising the IL-6R antagonist, or via ocular surface depots, punctual plugs or sub-palpebral depots.
